(19) United States
(12) Patent Application Publication
(10) Pub. No.: US 2026/0158510 A1
(43) Pub. Date: Jun. 11, 2026

(54) SIMPLIFIED ATOMIZER DEVICE

(71) Applicant: ADVANCED MEDICAL ENGINEERING, LLC, Sheridan, WY (US)

(72) Inventors: Jeff Phipps, Cedar City, UT (US); Scott Skeen, Cedar City, UT (US)

(21) Appl. No.: 19/393,409

(22) Filed: Nov. 18, 2025

Related U.S. Application Data
(60) Provisional application No. 63/721,971, filed on Nov. 18, 2024.

(51) Int. Cl. B05B 7/04; B05B 7/24

(52) U.S. Cl. CPC B05B 7/0458; B05B 7/2491

(57) ABSTRACT

An atomizer device for use in discharging a liquid into or on the body of the patient, the device having a head unit having a proximal end and a distal end, the proximal end configured to receive a plunger and to receive a gas flow through a gas inlet, wherein the plunger is configured to actuate a valve that opens to allow a gas to flow through a gas channel in the head unit. The atomizer device further includes a container receptacle coupled to the head unit configured to hold a liquid and an atomizer nozzle coupled to the distal end of the head unit and configured to mix the gas and the liquid together. In some designs, the atomizer device has an fixed extension member and an adjustable extension member with a wire to enable the clinician to shape the tip of the atomizer device.

### CROSS REFERENCE TO RELATED APPLICATIONS

This application claims priority from Provisional Application Ser. No. 63/721,971 filed on Nov. 18, 2024, which is hereby incorporated by reference in its entirety.

### FIELD OF THE INVENTION

The present invention relates generally to atomizer devices. More particularly, the present invention relates to a simplified, air-assisted atomizer device having a minimalistic design.

### BACKGROUND OF THE INVENTION

Atomizers have been employed in medical practice for decades as a means of delivering liquid medications in the form of a fine mist or spray. Early designs were simple mechanical devices that relied on the Venturi effect, where pressurized air passing through a narrow nozzle created a low-pressure zone that drew liquid from a reservoir and dispersed it as droplets. These early atomizers were commonly used for topical treatments and respiratory therapies, enabling medication to reach localized areas or the respiratory tract efficiently.

Over time, atomizer technology evolved to meet the growing demands of precision and safety in medical applications. Today, modern atomizers are widely used for administering anesthetics, antiseptics, and other therapeutic agents directly to mucosal surfaces or into body cavities. They provide controlled dosing and improved coverage compared to manual application methods. Despite these advancements, many current designs remain complex, incorporating multiple components that increase manufacturing costs and introduce potential failure points and attendant choking hazards. This complexity underscores the need for simplified, reliable atomizer devices that maintain effective atomization with a fine spray while also reducing risks associated with pressurized systems. Atomizer devices requiring a pressured gas source for operation may be made safer if efficient atomization can be achieved at a lower supply pressure.

In view of the foregoing, there remains a need for an atomizer device that simplifies construction while maintaining or improving performance. The present invention addresses these problems by providing a design with fewer components, enhanced safety, and compatibility with various gas sources, all while achieving efficient atomization at lower supply pressures.

### BRIEF SUMMARY OF THE INVENTION

The simplified atomizer device disclosed herein may feature a flexible wire tip either internally or externally, configured to be shaped during medical procedures to accommodate patient-specific anatomies within the ear, nose, throat, and other regions. The atomizer device may be compatible with standard oxygen ports and other sources of compressed gas. The atomizer device may include ergonomic supports such as loop or ring structures for the index finger and thumb to stabilize the atomizer device during actuation to apply medicament. For example, in some embodiments, an atomizer device includes a head unit having a proximal end and a distal end, the proximal end configured to receive a plunger and to receive a gas flow through a gas inlet, wherein the plunger is configured to actuate a valve that opens to allow a gas to flow through a gas channel in the head unit. The atomizer device also includes a container receptacle coupled to the head unit configured to hold a liquid, and an atomizer nozzle coupled to the distal end of the head unit and configured to mix the gas and the liquid together.

In some embodiments of the present disclosure, an atomizer device includes a head unit including a cap having an internal threaded cavity, a sidewall of the cap having a proximal end, a distal end, and a gas channel extending therebetween, the proximal end comprising a plunger housing in fluid communication with the gas channel, the plunger housing further including a gas inlet in fluid communication with the gas channel, and the distal end further including a liquid channel in fluid communication with the internal threaded cavity. The atomizer device further includes a valve needle slidably disposed within the gas channel such that a proximal end of the valve needle is positioned within plunger housing, a spring positioned within the gas channel between a distal end of the valve needle and the distal end of the head unit, a plunger slidably disposed within the plunger housing, a body of the plunger having flexible tabs that secure the plunger within the housing, and an outlet tube assembly having a gas outlet tube coupled to the gas channel at the distal end of the head unit, and a liquid outlet tube coupled to the liquid channel at the distal end of the head unit.

In some embodiments of the present invention, an atomizer device features a plunger housing configured to receive a plunger with flexible locking tabs that engage corresponding windows to provide secure, tool-free assembly and prevent accidental withdrawal. For reusable applications, the atomizer device may include a proximal outlet tube assembly with asymmetrical ports for correct alignment and a releasable coupling to a distal outlet tube assembly, enabling partial reuse of the rest of the atomizer device while minimizing cross-contamination.

In some embodiments of the present invention, a method for assembling an atomizer device includes disposing a valve needle into a gas channel of a head unit, inserting a spring into the gas channel such that the spring is positioned between the disposed valve needle and a distal end of the head unit, securing an outlet tube assembly to the gas channel and a liquid channel at the distal end of the head unit, disposing a plunger in a plunger housing until the flexible tabs pass completely into the open channels of the plunger housing thereby securing the plunger.

### BRIEF DESCRIPTION OF THE SEVERAL VIEWS OF THE DRAWINGS

In order that the above-recited features and other advantages of the invention are obtained and will be readily understood, a more particular description of the invention briefly described above will be rendered by reference to specific embodiments thereof, which are illustrated in the appended drawings. These drawings depict only typical embodiments of the invention and are not therefore to be considered to limit the scope of the invention.

FIGS. 1A and 1B are each perspective views of an atomizer device, in accordance with one or more representative embodiments of the present disclosure.

FIG. 2 is a perspective view of an atomizer device, in accordance with one or more representative embodiments of the present disclosure.

FIGS. 3A and 3B are respectively a side view and a cross-sectional top view of an atomizer device with a closed valve, in accordance with one or more representative embodiments.

FIGS. 4A and 4B are respectively a side view and a cross-sectional top view of an atomizer device with a partially open valve, in accordance with one or more representative embodiments of the present disclosure.

FIGS. 5A and 5B are respectively a side view and a cross-sectional top view of an atomizer device with a fully open valve, in accordance with one or more representative embodiments of the present disclosure.

FIGS. 6A and 6B are respectively a top view and a side cross-sectional view of an atomizer device, in accordance with one or more representative embodiments of the present disclosure.

FIG. 7 is an exploded view of an atomizer device, in accordance with one or more representative embodiments of the present disclosure.

FIG. 8 is a perspective view of an atomizer device with a flexible locking mechanism, in accordance with one or more representative embodiments of the present disclosure.

FIG. 9 is a perspective view of an atomizer device with a T-valve, in accordance with one or more embodiments of the present disclosure.

FIG. 10 is a perspective view of a portion of an atomizer device, in accordance with one or more representative embodiments of the present disclosure.

FIG. 11 is an exploded view of a reusable atomizer device, in accordance with one or more representative embodiments of the present disclosure.

FIG. 12 is a front, side exploded view of a disposable atomizer device, in accordance with one or more representative embodiments of the present disclosure.

FIG. 13 is a back, side exploded view of a disposable atomizer device, in accordance with one or more representative embodiments of the present disclosure.

FIG. 14 is a sectional view of an atomizer nozzle, in accordance with one or more representative embodiments of the present disclosure.

FIG. 15 is a perspective view of an atomizer device for specific use in the ear, nose, and throat, in accordance with one or more representative embodiments of the present disclosure.

FIG. 16 is a perspective view of an atomizer device, in accordance with one or more representative embodiments of the present disclosure.

FIG. 17 is an exploded view of an atomizer device, in accordance with one or more representative embodiments of the present disclosure.

FIG. 18 is an enlarged view of an adjustable extension member with an internal wire, in accordance with one or more representative embodiments of the present disclosure.

FIG. 19 is a perspective view of an atomizer device, in accordance with one or more representative embodiments of the present disclosure.

FIG. 20 is an enlarged view of an adjustable extension member with an external wire, in accordance with one or more representative embodiments of the present disclosure.

### DETAILED DESCRIPTION OF THE INVENTION

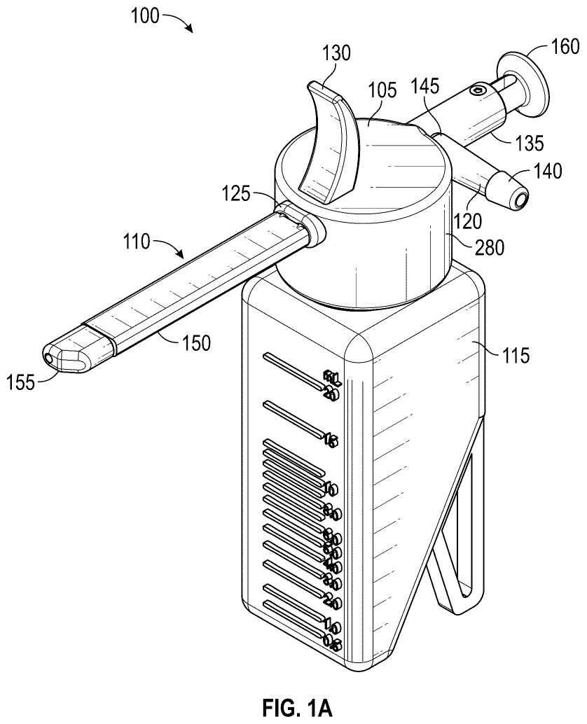

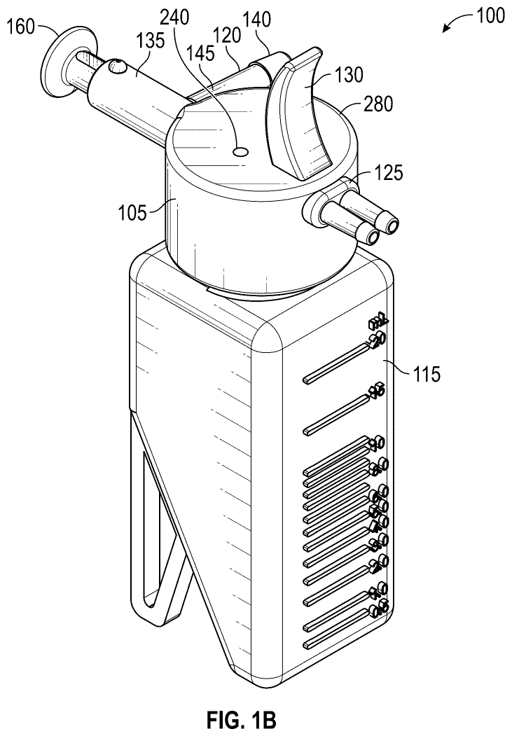

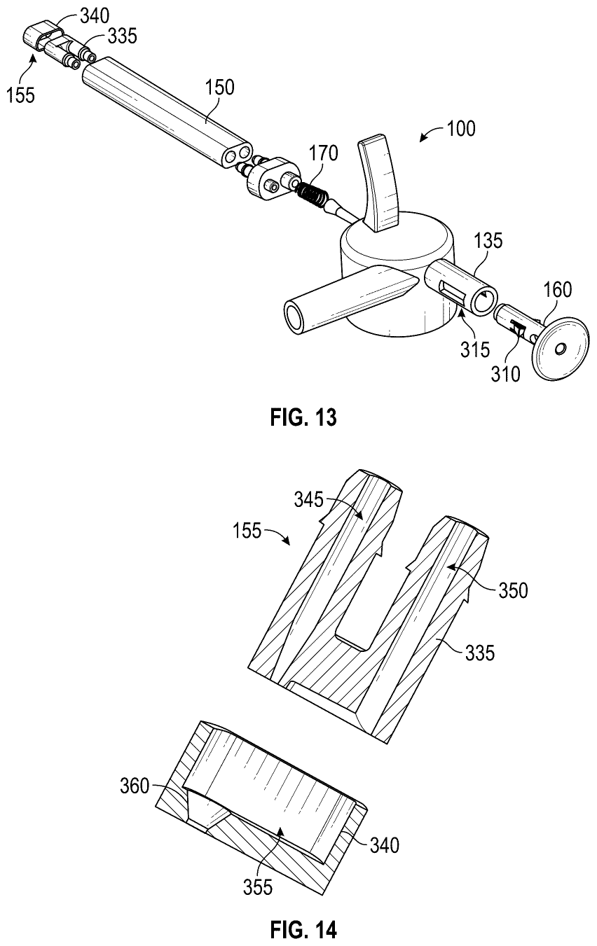

Embodiments of the present disclosure will be best understood by reference to the figures, wherein like reference numbers indicate identical or functionally similar elements. It will be readily understood that the components of the present disclosure, as generally described and illustrated in the figures, could be arranged and designed in a wide variety of different configurations. Thus, the following more detailed description, as represented in FIGS. 1 through 20, is not intended to limit the scope of the invention as claimed but is merely representative of some presently preferred embodiments of the invention.

The present disclosure describes apparatuses, systems, and methods for providing a simplified atomizer device with ergonomic features. The atomizer device disclosed herein may include the ability to place the valve axis at or near a central axis of the head unit for ambidextrous usage or at any position offset from the central axis for convenient operation by a user's dominant hand. The atomizer device disclosed herein includes fewer components, can be smaller and more compact, is simpler to manufacture and assemble (with design features preventing incorrect assembly), and contains fewer break or fail points, which can help prevent broken atomizers which can injure a patient. Additionally, the ergonomic support disclosed herein adds to the comfort and usability of the atomizer device, which can lead to increased accuracy and precision for practitioners who spray medicament into or onto a patient. The variable valve disclosed herein also contributes to the generation of a fine, uniform spray from the atomizer device.

FIGS. 1A and 1B are each a perspective view of a simplified atomizer device 100, according to a representative embodiment of the present disclosure. The atomizer device 100 may include a head unit 105, nozzle component 110, and a container 115. In some embodiments, the head unit 105 includes a cap 280, a gas inlet 120, an outlet tube assembly 125, a finger support 130, and a plunger housing 135, which, in some embodiments are integrally formed severally or entirely as a monolithic structure. In some instances, head unit 105 and/or atomizer device 100 are intended to be disposable single-use atomizer devices. A disposable atomizer in medical settings reduces the risk of cross-contamination between patients, ensuring a higher standard of hygiene and infection control. Additionally, it eliminates the need for cleaning and sterilization, saving time and resources while improving patient safety.

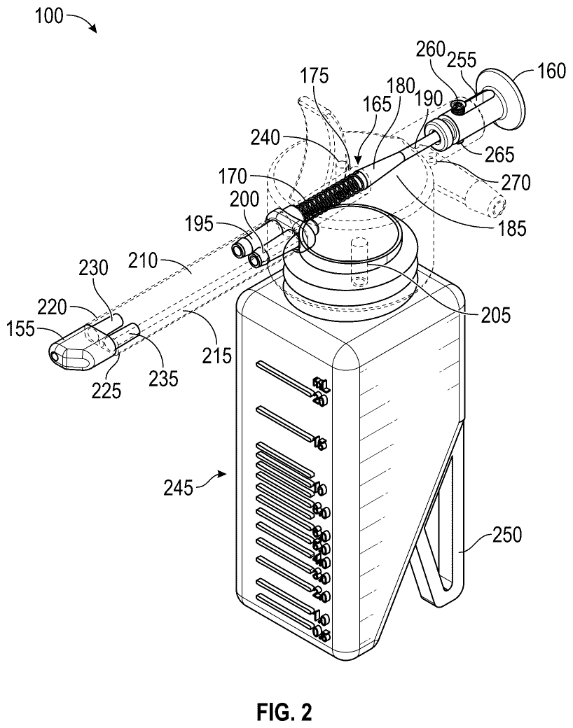

The cap 280 of head unit 105 is generally cylindrical with a flat top surface, from where finger support 130 extends upwards. The top and bottom edges of the cap 280, the edges of finger support 130, and any other edges of head unit 105 may be chamfered to reduce sharp edges. Cap 280 of head unit 105 may include an internal cavity configured to receive container 115, such as by a threaded connection or a quarter-turn attachment interface to ensure alignment and sealing of the container 115. Cap 280 may also include a sidewall that has a proximal end, a distal end, and a gas channel 175 (as shown in FIG. 2) extending therebetween. In some embodiments, the proximal end of cap 280 includes the plunger housing 135 which is in fluid communication with the gas channel 175. The distal end of the cap 280 may include a liquid channel 205 in fluid communication with the head unit 105, outlet tube assembly 125, and nozzle component 110. The outlet tube assembly may be attached to the head unit permanently or temporarily via any suitable attachment mechanism, including sonic welding, glue, standard or customized threading, elastic restraints, one or more flexible supports (i.e., live hinges) fixed to the outlet tube assembly 125 or head unit, or any other conceivable manner. In some embodiments, container 115 includes a fluid reservoir in which is stored a medicament or other liquid which is intended to be administered to a patient via atomizer device 100.

In some embodiments, plunger housing 135 includes gas inlet 120 with a lumen that passes therethrough for establishing fluid communication with plunger housing 135 and gas channel 175. Gas inlet 120 is configured to be coupled with a source of pressurized gas (not shown). In some embodiments, the pressurized gas is medical air, oxygen, nitrous oxide, carbon dioxide, nitrogen, or helium. In some embodiments the pressurized gas may be supplied by the available building facilities, a portable or semi-portable fixed volume compressed gas container, a handheld battery powered device, or a user-powered compressed gas device such as a handheld pump. Gas inlet 120 includes a distal end 140 having features for retaining a pressurized gas line. Gas inlet 120 also includes a proximal end 145 that can be permanently or temporarily coupled to head unit 105. In some embodiments, proximal end 145 of gas inlet 120 is coupled to plunger housing 135 and is adjacent to the main body of head unit 105. The position of the gas inlet may be angled relative to the top flat surface of the head unit 105 and at any distance from the head unit.

The nozzle component 110 may include a nozzle extension 150 and atomizer nozzle 155. Nozzle extension 150 may be optional in some embodiments. In some embodiments, nozzle extension 150 is rigid. In other embodiments, nozzle extension 150 is flexible, or semi-flexible thereby enabling a user to position or reposition nozzle extension 150 to achieve and retain a desired configuration. In some embodiments, nozzle extension 150 may be bendable and include a lumen for securing a wire. The wire may be embedded within the lumen along a length of the nozzle extension 150, providing structural support while also allowing a user to adjust a spray direction as needed. The wire may have malleable properties such that when formed, bent, or shaped, the wire will retain the same or similar position without rebounding back to a straight or previous position. In another embodiment, the tubing material may itself have malleable properties and an ability to retain its position without additional support. In another embodiment, the tubing comprises or is enclosed within a segmented tube having a plurality of articulating pieces to permit the previously described adjustability. In another embodiment the atomizer nozzle 155 position can be controlled remotely by a mechanism positioned near or incorporated into the finger support 130.

The plunger housing 135 may be configured to slidably receive a plunger 160, which when pressed in a distal direction, opens a valve (e.g., valve 165 shown in FIG. 2) which allows gas to pass through gas inlet 120, gas channel 175 of head unit 105, nozzle extension 150 and out atomizer nozzle 155.

Finger support 130 extends upwards from and is fixed to the top surface of head unit 105. Finger support 130 may be integrally formed with head unit 105. Finger support 130 may be curved and designed to accommodate a finger (i.e., an index finger) comfortably while a user holds atomizer device 100 with one hand. The finger support may also be configured as a ring or another enclosed or semi-enclosed shape through which the user's finger may be fitted to prevent accidental release of the device. The user's thumb may rest comfortably on a proximal end of plunger 160. The rest of the user's fingers may wrap around container 115, below nozzle extension 150, thus providing an ergonomic and comfortable hold on atomizer device 100. In some embodiments, the container 115 or head unit 105 may include one or more ergonomic features to further enhance the comfort and control of the device during use. Additionally, when applying a force on plunger 160 to move it in a distal direction and open valve 165, the user's index finger may apply a counterforce to finger support 130, making it easier to apply the force to plunger 160.

FIG. 2 is another perspective view of atomizer device 100, in accordance with one or more embodiments of the present disclosure. FIG. 2 shows additional internal details for atomizer device 100. For example, within head unit 105 of atomizer device 100, a valve 165 may be positioned within gas channel 175 that passes horizontally through head unit 105.

Valve 165 may include a valve needle 190 slidably disposed within gas channel 175 such that a proximal end of valve needle 190 is positioned within plunger housing 135. Valve needle 190 may include a tapered outer surface 180 that is configured to contact a tapered inner surface 185 of gas channel 175 to selectively prevent a gas from flowing through gas channel 175. A proximal end of valve needle 190 may be coupled to or in contact with plunger 160. A distal end of valve needle 190 may be coupled to or in contact with a spring 170. Tapered outer surface 180 may include any angle. In some embodiments, an angle of tapered outer surface 180 is selected to achieve desired gas flow and/or flow dynamics of a pressurized gas flowing through the atomizer device.

Spring 170 is positioned within gas channel 175 between a distal end of valve needle 190 and the distal end of head unit 105. Spring 170 is biased such that it forces valve needle 190 in a proximal direction when no external force is applied (i.e., distal force applied to plunger 160). Spring 170 may be compressed and retained between valve needle 190 and outlet tube assembly 125. Specifically, the distal end of spring 170 is in contact with or coupled to outlet tube assembly 125 or, in some embodiments, a distal portion of sidewall of cap 280 of head unit 105.

In some embodiments, outlet tube assembly 125 is in fluid communication with gas channel 175 and liquid channel 205. Outlet tube assembly 125 may serve as a fitting that retains spring 170 and provides isolated connection points between gas channel 175 and liquid channel 205 and nozzle extension 150 and atomizer nozzle 155. In one or more embodiments, outlet tube assembly 125 is secured within head unit 105 by sonic welding or by applying adhesive between outlet tube assembly 125 and head unit 105. In other embodiments, outlet tube assembly 125 is selectively coupled to head unit 105 via at least one of an interference fit, a snap fit, a threaded interface, and a latch and catch engagement. In some instances, a base of the outlet tube assembly comprises a catch or other compatible surface configured to temporarily receive a latch flexibly or semi-flexibly coupled to the head unit. In some instances, a base of the outlet tube assembly comprises one or more flexible latches having a terminal end configured to engage one or more catches, grooves, shelves, rims, or other compatible surfaces provided on the head unit in proximity to the outlet tube assembly. Outlet tube assembly 125 may include or be coupled to two separate tube structures, a gas outlet tube 195 and liquid outlet tube 200, each extending away from head unit 105 in a distal direction. Gas outlet tube 195 is in fluid communication with gas channel 175 while liquid outlet tube 200 is in fluid communication with liquid channel 205. A distal end of gas outlet tube 195 is coupled to, and in fluid communication with, a gas extension tube 210, while a distal end of liquid outlet tube 200 is coupled to, and in fluid communication with, a liquid extension tube 215. Gas extension tube 210 and liquid extension tube 215 may be housed within nozzle extension 150. In some embodiments, gas outlet tube 195 has a larger diameter than liquid outlet tube 200 or vice versa.

Gas extension tube 210 and liquid extension tube 215 include distal ends 220 and 225, respectively, which terminate at and are received by atomizer nozzle 155. Specifically, atomizer nozzle 155 includes gas receiver tube 230 and liquid receiver tube 235, wherein gas receiver tube 230 receives and is coupled to distal end 220 of gas extension tube 210, and wherein liquid receiver tube 235 receives and is coupled to distal end 225 of liquid extension tube 215. As the velocity of a gas increases through atomizer nozzle 155, pressure within liquid extension tube 215 decreases thereby drawing liquid from container 115, through liquid channel 205 and liquid outlet tube 200, and ultimately into atomizer nozzle 155, where the liquid and gas mix to form the desired atomized solution. Upon release or deactivation of plunger 160, which closes valve 165, the gas flowing through gas extension tube 210 ceases, which returns the pressure within liquid extension tube 215 to atmospheric pressure, which stops the movement of liquid out of container 115. In some embodiments, the liquid extension tube 215, the liquid side of the outlet tube assembly 125, or the space between outlet tube assembly 125 and the liquid channel 205 may include a check valve to prevent back flow of the liquid into the container.

Head unit 105 may include a vent 240 which provides an airway and facilitates pressure equilibrium between the fluid reservoir of container 115 and an exterior environment. Therefore, when valve 165 is open and liquid is flowing out of container 115, vent 240 prevents negative pressure buildup within container 115, thus preventing a vacuum in container 115 which would cause a loss of liquid flow. Further, in some embodiments vent 240 includes a filter to prevent contamination of a medicament or other liquid stored in the fluid reservoir of container 115.

In some embodiments, liquid channel 205 of head unit 105 extends into the internal cavity of cap 280 and is configured to receive and couple to a pickup tube (not shown) for retrieving a liquid from container 115 when container 115 is coupled to head unit 105. The pickup tube extends outwardly from head unit 105 and into container 115. Thus, a fluid within container 115 is withdrawn via the pickup tube and delivered to liquid channel 205 and ultimately to atomizer nozzle 155.

In some embodiments, container 115 includes a threaded portion that extends upwards from a top planar surface of container 115. The threaded portion may include exterior threads that extend outward from the threaded portion and correspond and are sized to fit interior threads formed on a hollow surface of the main body of head unit 105. The threads may be designed such that container 115 always faces a predetermined direction following full engagement of the threads. In another embodiment the container 115 and head unit 105 may be connected by non-threaded means, such as, for example a keyed interface. In one embodiment, the container 115 and head unit 105 may be coupled in any suitable manner including via a snap fit, friction fit, interference fit, etc. The top planar surface may be generally rectangular or square-shaped. About half-way down from one side (e.g., a proximal side), an internal wall structure of container 115 is sloped such that liquid in container 115 is concentrated in a smaller surface area than the top of container 115. This structure enables the pickup tube to draw nearly all the remaining contents as liquid levels decrease, minimizing waste and leading to a complete evacuation of the container. Container 115 includes markings 245 (e.g., graduation marks or measurement indicators) to indicate an amount of liquid contained or remaining in container 115. In some embodiments, markings 245 include a series of lines spaced at defined intervals along a side of container 115, and include numerical volume unit labels (e.g., in milliliters or ounces). Container 115 may include one or more support legs 250 which have a ground contact surface at a same level as the bottom surface of container 115. Support legs 250 may include any shape and are configured to support atomizer device 100 such that atomizer device 100 can stand upright on its own when coupled with container 115. Support legs 250 may also provide additional support for a user's hand when holding the atomizer device 100 adding to additional ergonomics for atomizer device 100. In one embodiment, support legs 250 are generally triangular in shape.

Plunger housing 135 includes a hollow center that is configured and sized to receive plunger 160. Plunger 160 may be slidably disposed within plunger housing 135. The body of plunger 160 may include a slot (e.g., an elongated through-hole) 255 extending along a length of the body of plunger 160. Slot 255 may be sized to allow a securement pin 260 to be slidably disposed within slot 255. Securement pin 260 may be inserted through plunger housing 135 and the slot of plunger 160 such that securement pin 260 retains plunger 160 within plunger housing 135. Securement pin 260 is fixed to plunger housing 135 via any suitable mechanism. For example, securement pin 260 may be fixed to plunger housing 135 via threads. The threads may be near the head of the securement pin 260, which are received by corresponding threads on a top of plunger housing 135. In one or more embodiments, the threads are at a bottom of securement pin 260, furthest away from the head of securement pin 260, and are received by corresponding threads on a bottom of plunger housing 135. Securement pin 260 may be inserted into slot 255 after plunger 160 is inserted into plunger housing 135. In some embodiments, securement pin 260 is held in place with a nut on an opposite (e.g., bottom) side of plunger housing 135. In some embodiments, securement pin 260 is held in place with adhesive or compressible expanding tabs. The compressible expanding tabs are designed to compress as the pin slides through the plunger housing 135 and plunger 160 and expand once the securement pin is in place. In some embodiments, securement pin 260 is permanently secured in place. In some embodiments, securement pin 260 is selectively secured in place. Plunger housing 135 may include an annular groove 265, which can secure an optional O-ring to seal the hollow opening in plunger housing 135 from the exterior environment. A distal end of plunger 160 may include a hollow blind bore that is sized and configured to receive a proximal end of valve needle 190 preventing valve needle 190 from moving around and keeping valve needle 190 centered and coaxially aligned with plunger 160 and within plunger housing 135 and gas channel 175.

Plunger housing 135 may include one or more apertures 270 in fluid communication with gas inlet 120 when plunger 160 is in an extended (i.e., proximal) position. In such an extended position, aperture 270 provides an outlet for gas entering the hollow center of plunger housing 135 via gas inlet 120. Thus, when a pressurized gas line is coupled to gas inlet 120, pressure does not build up in the hollow center of plunger housing 135. When plunger 160 is moved distally, a distal end of plunger 160 covers and seals aperture 270 such that gas flowing from gas inlet 120 can no longer escape via aperture 270, but is forced past the now-open valve 165 and into gas channel 175.

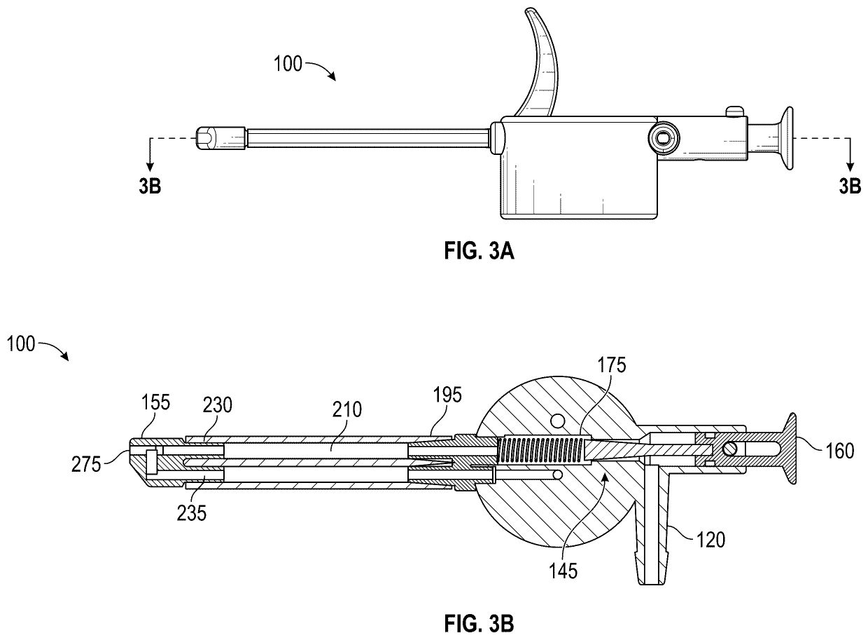

FIGS. 3A and 3B are respectively a side view and a cross-sectional top view of atomizer device 100 with a closed valve 165, in accordance with one or more embodiments. As shown in FIG. 3B, when no distal force is applied to plunger 160, spring 170 forces tapered outer surface 180 of valve needle 190 against tapered inner surface 185 of gas channel 175, causing valve 165 to close, disallowing any gas from gas inlet 120 to pass through to gas channel 175. Spring 170 is sufficiently biased such that a strong seal is formed between tapered outer surface 180 and tapered inner surface 185.

FIGS. 3B, 4B, and 5B show how atomizer device 100 uses the venturi effect to produce a fine mist when valve 165 is partially or fully open. A user actuates plunger 160, which in turn opens valve 165, allowing gas to flow from gas inlet 120, through gas channel 175, out gas outlet tube 195, through gas extension tube 210, through gas receiver tube 230, and out atomizer nozzle 155. Atomizer nozzle 155 includes a single nozzle outlet 275 in fluid communication with two inlets (e.g., gas receiver tube 230 and liquid receiver tube 235). A lumen connected to liquid receiver tube 235 connects to a lumen connected to gas receiver tube 230 at a 90-degree angle. As gas flows through the lumen connected to gas receiver tube 230, a drop in pressure is experienced in the liquid flow lines, which causes a suction effect drawing liquid out of container 115. The drawn-out liquid is mixed with the gas as both the liquid and gas exit the nozzle outlet 275 simultaneously, atomizing the liquid into tiny droplets and producing a fine dispersed mist.

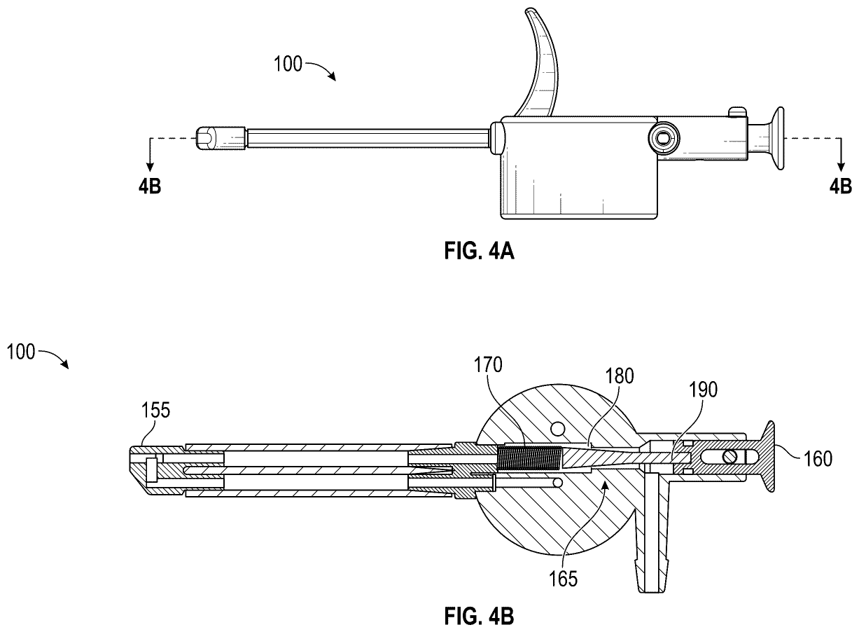

FIGS. 4A and 4B are respectively a side view and a cross-sectional top view of atomizer device 100 with a partially open valve 165, in accordance with one or more embodiments of the present disclosure. When a distal force is applied to plunger 160, the force is transferred in turn to valve needle 190, overcoming the biased counterforce of spring 170. A practitioner using atomizer device 100 may desire to only spray a small amount of medicament, and therefore may only desire to open the valve partially, which allows a smaller amount of gas through to the atomizer nozzle 155. Moreover, during partial opening of the valve, the pressure relief aperture 270 remains only partially restricted thereby preventing excessive pressure build-up in the plunger housing 135 and enabling finer control of gas flow to the atomizer nozzle 155. A smaller volume of gas passing through atomizer nozzle 155 will produce a smaller volume of liquid drawn and subsequently sprayed. Thus, by controlling how much force is applied to plunger 160, a practitioner can control how much medicament is applied to a patient.

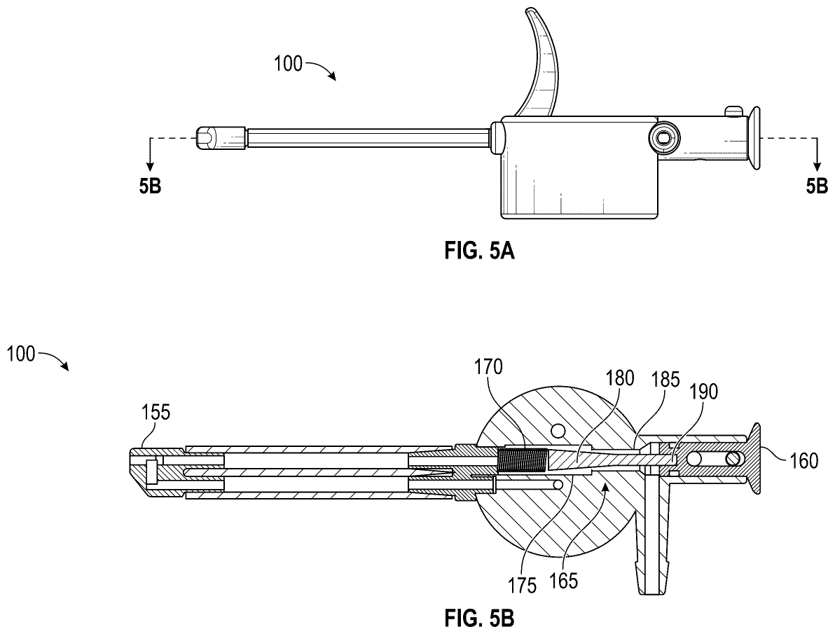

FIGS. 5A and 5B are respectively a side view and a cross-sectional top view of atomizer device 100 with a fully open valve 165, in accordance with one or more embodiments of the present disclosure. When a sufficient force is applied to plunger 160 such that spring 170 is fully compressed, a larger gap appears between valve needle 190 and tapered inner surface 185 of gas channel 175, thus allowing the maximum volume of gas to pass through gas channel 175. When spring 170 is fully compressed, and the valve 165 is fully open, the maximum amount of spray volume will be emitted from atomizer nozzle 155. In other words, the amount of spray volume that is emitted from atomizer nozzle 155 is generally proportional to the force applied to the plunger 160.

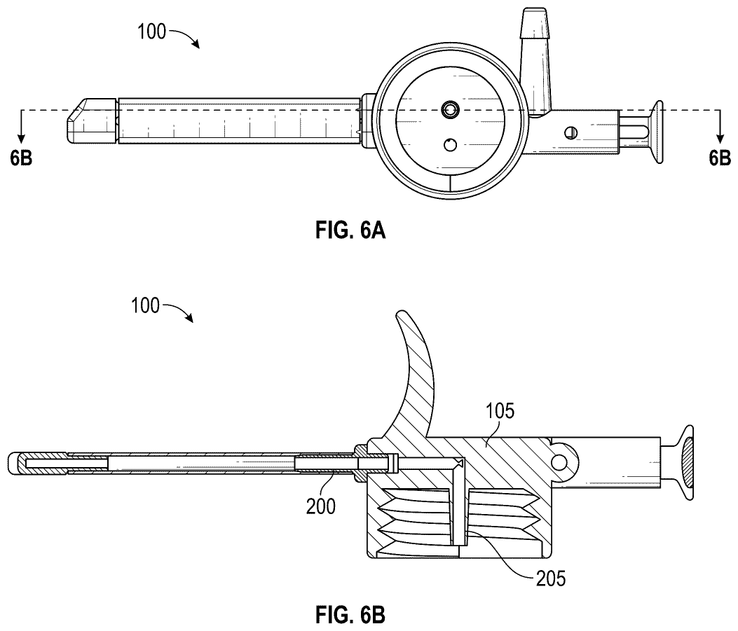

FIGS. 6A and 6B are respectively a top view and a side cross-sectional view of atomizer device 100, in accordance with one or more embodiments of the present disclosure. Head unit 105 includes liquid channel 205 in fluid communication with liquid outlet tube 200. In some embodiments, liquid channel 205 extends and protrudes vertically downwards from the internal cavity. In some embodiments, liquid channel 205 does not extend past a lowest point of head unit 105. Liquid channel 205 may extend into head unit 105 and turn at a 90-degree angle to connect to liquid outlet tube 200. In some embodiments, a pickup tube (not shown) is coupled to liquid channel 205, and extends to a bottom of container 115. In some embodiments the liquid channel extends to, or within a few millimeters of, the bottom of the container 115 such that a detachable pickup tube is unnecessary.

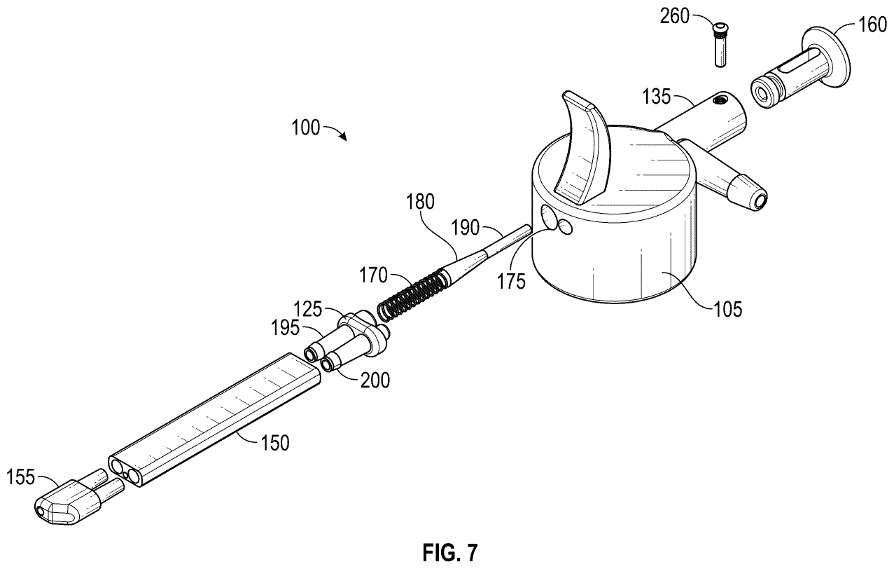

FIG. 7 is an exploded view of atomizer device 100, in accordance with one or more embodiments of the present disclosure. Atomizer device 100 may be assembled by combining all the components shown in FIG. 7. Head unit 105 may be formed initially using any suitable technique. For example, in some embodiments, head unit 105 is formed using injection molding, blow molding, 3D printing, or casting. Plunger 160 may then be inserted into plunger housing 135 of head unit 105 and secured with securement pin 260. Valve needle 190 may be disposed and/or inserted into gas channel 175. A proximal end of valve needle 190 may be pressed against plunger 160. Plunger 160 receives valve needle 190 in the blind bore at the distal end of the body of plunger 160. Spring 170 may then be inserted into gas channel 175 and pressed against a distal end of the valve needle 190.

Outlet tube assembly 125 may in some embodiments include gas outlet tube 195 and liquid outlet tube 200. Outlet tube assembly 125 may then be coupled to head unit 105 by any suitable manner. For example, an adhesive may be applied to a proximal end of outlet tube assembly 125 which is pressed against head unit 105 until a secure attachment is formed. In some embodiments, gas outlet tube 195 and liquid outlet tube 200 are integrally formed with outlet tube assembly 125 as a monolithic unit. In other embodiments, gas outlet tube 195 and/or liquid outlet tube 200 are not integrally formed with outlet tube assembly 125 and can be coupled to outlet tube assembly 125 and/or head unit 105 individually in any suitable manner. For example, in some embodiments, each of gas outlet tube 195 and liquid outlet tube 200 are coupled to outlet tube assembly 125 and/or head unit via threads, an interference fit, a latch and catch engagement, one or more flexible retaining members, or a snap fit. In some embodiments, any of these manners of coupling may be secured with adhesive or by sonic welding.

In some embodiments, liquid outlet tube 200 and/or outlet tube assembly 125 (without gas outlet tube 195) is integrally formed with head unit 105 as a monolithic unit. In this embodiment, valve needle 190 and spring 170 are inserted and then gas outlet tube may be coupled to head unit 105 via any suitable manner. For example, gas outlet tube 195 is coupled to outlet tube assembly 125 and/or head unit 105 via threads, an interference fit, a latch and catch engagement, one or more flexible retaining members, or a snap fit, or via any other suitable manner. Adhesives or sonic welding may be applied to secure such couplings.

Outlet tube assembly 125 may be offset from a center axis of head unit 105. In some embodiments, gas channel 175, and therefore gas outlet tube 195, is aligned with a center of the main body of head unit 105. In such embodiments, since gas outlet tube 195 is aligned with a center of the main body of head unit 105, liquid outlet tube 200 is off-center. In some embodiments, gas outlet tube 195 and liquid outlet tube 200 flank a center axis of the main body of head unit 105.

Following the coupling of the outlet tube assembly 125 to head unit 105, nozzle extension 150 can optionally be fitted to the outlet tube assembly 125 in any suitable manner. For example, in some embodiments, nozzle extension 150 is fitted to outlet tube assembly 125 via an interference fit, a snap fit, a press fit, a threaded connection, a clamped or crimped connection, adhesive bonding, sonic welding, etc.

Atomizer nozzle 155 may then be coupled to nozzle extension 150 in any suitable manner. For example, atomizer nozzle 155 may be coupled and/or fitted to nozzle extension 150 via an interference fit, a snap fit, a press fit, a threaded connection, a clamped or crimped connection, adhesive bonding, sonic welding, etc.

While assembly is described in this disclosure in a particular sequence or order of steps, a different order of steps may be taken. For example, atomizer nozzle 155 may be coupled to nozzle extension 150 before nozzle extension is coupled to outlet tube assembly 125. Similarly, atomizer nozzle 155, nozzle extension 150 and outlet tube assembly 125 may be assembled and coupled together before coupling to head unit 105.

In some embodiments, valve needle 190 is disposed into gas channel 175 of head unit 105. Spring 170 may then be inserted into gas channel 175 such that spring 170 is positioned between the disposed valve needle 190 and the distal end of head unit 105. Outlet tube assembly may be secured to gas channel 175 and liquid channel 205 at the distal end of head unit 105. Plunger 160 may be disposed in plunger housing 135. Plunger 160 may temporarily be held in a depressed position within plunger housing 135 while securement pin 260 is secured through plunger housing 135 and slot 255 of depressed plunger 160. Depressed plunger 160 may then be released after securement pin 260 is secured to plunger housing 135. Nozzle extension 150 may then be coupled to outlet tube assembly 125. In some embodiments, nozzle extension 150 includes a wire such that that nozzle extension 150 is configured to retain a bent configuration. The wire may have malleable properties such that when formed, bent, or shaped, the wire will retain the same or similar position without rebounding back to a straight or previous position. In another embodiment, the tubing material may itself have malleable properties and an ability to retain its position without additional support. In another embodiment, the tubing is constructed of, or enclosed in, a series of segmented pieces with ball-and-socket style joints at each connection point allowing for the previously described adjustability. In another embodiment the atomizer tip 155 position can be controlled remotely by a mechanism near the finger support 130. Such a mechanism allows adjustment of the atomizer tip angular position without the use of a second hand and/or while the atomizer tip is inserted into the patient. In one or more embodiments, outlet tube assembly 125 includes nozzle extension 150 and atomizer nozzle 155.

As shown in FIG. 7, atomizer device 100 includes a minimal number of individual pieces, which reduces complexity of assembly and use. Minimal pieces also reduce probability of failure of the device since any piece could potentially be a point of failure, fewer number of pieces leads to a lower potential for failure. Features are also designed to prevent incorrect assembly. Further, fewer components increase safety since a greater number of components could lead to breakage of each component. When a component of an atomizer sprayer used in a medical setting breaks, the device may be near a patient's face, which could cause injury to the patient's face or body. The atomizer device 100 is further designed to operate efficiently at gas supply pressures lower than the supply pressures required by existing devices making it less likely to fail and cause patient injury.

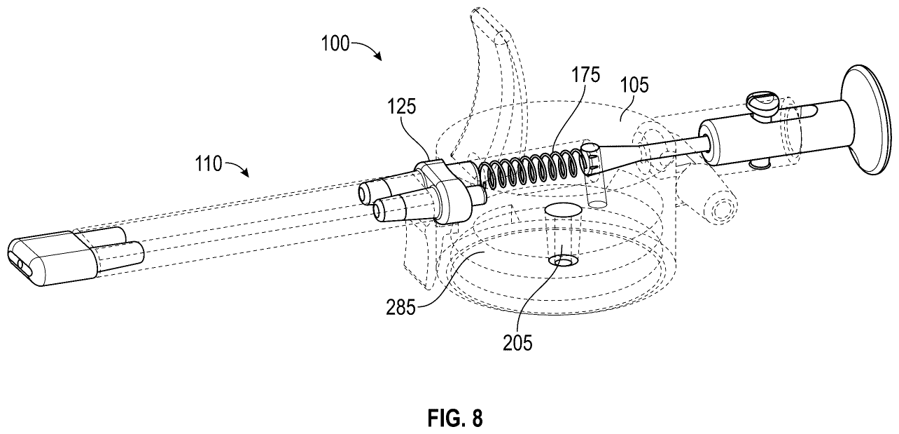

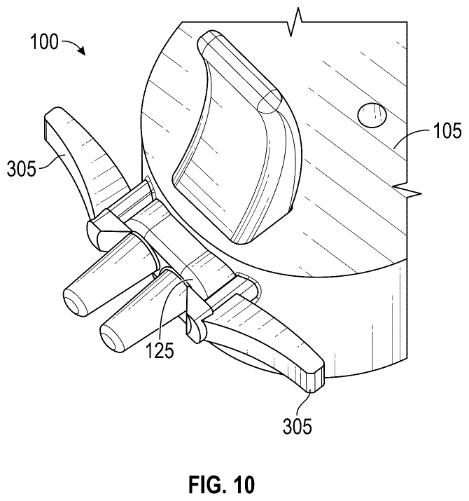

FIG. 8 is a perspective view of atomizer device 100 with a flexible locking mechanism 285, in accordance with one or more embodiments of the present disclosure. In other embodiments two or more flexible locking mechanisms may be used above and below the outlet tube assembly or on either side (as shown by flexible locking mechanism tabs in FIG. 10). Locking mechanism 285 or other embodiments thereof may be configured such that outlet tube assembly 125 and nozzle component 110 can be removably coupled to head unit 105. In some embodiments, locking mechanism 285 may be fixed to a distal end of head unit 105 below and/or above outlets for the gas channel 175 and the liquid channel 205. In one or more embodiments, locking mechanism 285 may be integrally formed with head unit 105 as part of a monolithic structure formed during injection molding, 3D printing, casting, etc. of head unit 105. The locking mechanism or mechanisms may include a tab or tabs that extend(s) outwards away from head unit 105, a lever or levers that extends downwards or in any direction away from a distal end of the tab, and a lip or lips that extends substantially upwards or inwards from the tab. The lip(s) is/are configured to engage outlet tube assembly 125 and prevent outlet tube assembly 125 from moving in a distal direction, thus securing the outlet tube assembly 125 in gas channel 175 and liquid channel 205 against a distal end of head unit 105. The locking mechanism may be constructed of a flexible, pliable material that allows a user to actuate (e.g., push) on the lever(s) to release outlet tube assembly 125. For example, pushing on the lever of locking mechanism 285 may lower the lip, disengaging the lip from outlet tube assembly 125 and allowing it to move in a distal direction. In an embodiment, outlet tube assembly 125 is not secured to head unit 105 with any other mechanism other than locking mechanism 125, which allows outlet tube assembly 125 to slide in or out of gas channel 175 and liquid channel 205 when locking mechanism 285 is actuated. In this manner, outlet tube assembly 125 and nozzle component 110 can easily be removed and discarded while head unit 105 may be subsequently reused. In other embodiments the device may include elastic restraints or tapered inserts that tighten the outlet tube assembly 125 against the head unit as the insert is pressed into place.

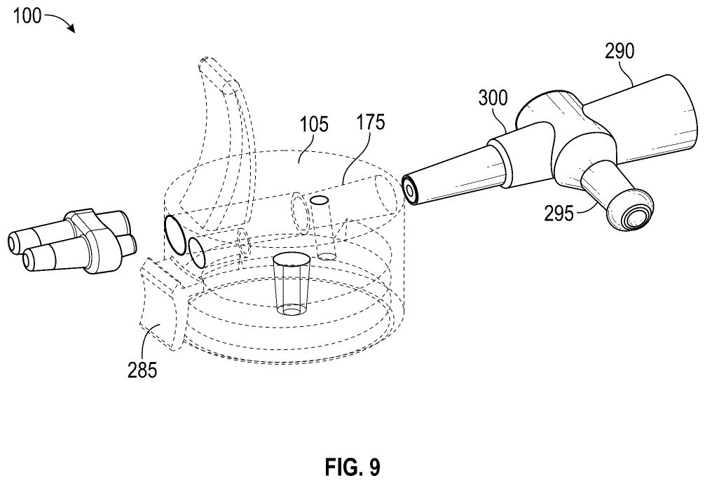

FIG. 9 is a perspective view of atomizer device 100 with a T-valve 290, in accordance with one or more embodiments of the present disclosure. In some embodiments, when T-valve 290 is used, plunger housing 135, plunger 160, gas inlet 120, and valve 165 are not used in atomizer device 100. T-valve 290 may include gas inlet arm 295 and main arm 300 which may intersect each other and each be at least partially hollow. In some embodiments, gas inlet arm 295 and main arm 300 are in fluid communication. Main arm 300 may include a hollow center that passes completely through from a proximal end to a distal end. Gas inlet arm 295 may include a hollow center but is open on one side and closed on the other. Gas inlet arm 295 may include features to receive and retain a pressurized gas line. Main arm 300 may be inserted into gas channel 175 and may be retained in gas channel 175 via any suitable mechanism. For example, main arm 300 may be retained in gas channel 175 via an interference fit, a friction fit, or a snap fit. In some embodiments, an adhesive is applied to a distal end of main arm 300 before inserting into gas channel 175 to create a more permanent fitting.

A practitioner may hold atomizer device 100 with T-valve 290 in much the same manner as atomizer device 100 with plunger housing 135 and plunger 160. However, in the embodiment shown in FIG. 9, gas flowing in from gas inlet arm 295 is directed out the proximal end of main arm 300 since the proximal end of main arm 300 is left open. The practitioner may then cover the proximal end of main arm 300 with their thumb, which seals the proximal end of main arm 300 and forces the pressurized gas flowing in through gas inlet arm 295 to travel through the distal end of main arm 300 and through gas channel 175, ultimately exiting atomizer device via atomizer nozzle 155 (not shown in FIG. 9). Once the practitioner releases their thumb from being pressed against the proximal end of main arm 300, the pressure in gas channel 175 drops and the gas flows freely out of the proximal end of main arm 300. In this manner, a conventional and oft-used T-valve may be used with head unit 105 that has been slightly modified from head unit 105 shown in FIGS. 1-8.

FIG. 10 is a perspective view of a portion of atomizer device 100, in accordance with one or more embodiments of the present disclosure. Flexible locking mechanism tabs 305 may be positioned on a distal end of head unit 105 on one or both sides, or above and/or below the openings in head unit 105 for gas channel 175 and liquid channel 205. In some embodiments, flexible locking mechanism tabs 305 are configured to receive and secure outlet tube assembly 125 and function in a similar manner as flexible locking mechanism 285 shown in FIGS. 8 and 9.

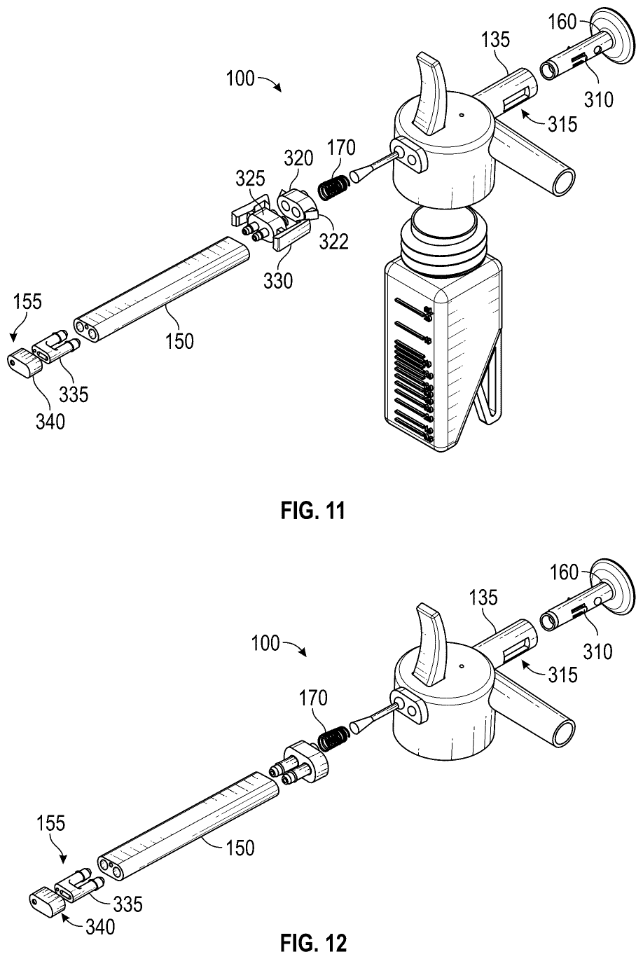

FIG. 11 is an exploded view of atomizer device 100, intended to be reusable, in accordance with one or more embodiments of the present disclosure. As shown therein, atomizer device 100 may include a plunger housing 135 configured to slidably receive a plunger 160. In some embodiments, the plunger 160 may have a pair of flexible, angled tabs 310 projecting laterally from opposing sides thereof. Each of the tabs 310 may include an outwardly facing mating surface and an inwardly sloped lead-in surface with a flat back edge such that the tabs 310 flex inward during insertion due to the angled portion but cannot be easily removed due to the flat back edge. The plunger housing 135 may define corresponding windows 315 (e.g., slots or elongated through-holes) in its sidewalls, each of the windows 315 being sized and positioned to receive a respective tab 310 from the plunger 160. During insertion of the plunger 160 into the plunger housing 135, the angled lead-in surfaces of the tabs 310 engage the edges of the windows 315, causing the tabs 310 to resiliently deflect inward. Upon reaching a fully seated position, the tabs 310 spring outward so that their mating surfaces extend through the windows and bear against the exterior surface of the plunger housing 135, thereby forming an interlock that resists axial withdrawal of the plunger 160. It will be appreciated that this alternative configuration provides a secure mechanical engagement without requiring separate fasteners such as the securement pin discussed earlier. The reduction in overall component pieces like the securement pin mitigates against points of failure and simplifies use of the atomizer 100. Other locking mechanisms, including without limitation, interference fit, friction fit, snap fit, threads, sonic welds, etc. may additionally or alternatively be used.

Functionally speaking, and as depicted earlier in FIG. 2, the plunger 160 may then be pressed in a distal direction, opening the valve 165 which allows gas to pass through gas inlet 120, gas channel 175 of head unit 105, nozzle extension 150 and out atomizer nozzle 155. To remove the plunger 160 from the plunger housing 135, the tabs 310 are manually deflected inward to clear the mating engagement with the edges of the windows 315. This may be accomplished by applying inward pressure to the tabs 310 through a pinching gesture with the fingers or use of a suitable tool. Once the tabs 310 are displaced sufficiently to disengage from the windows 315, the plunger 160 can be withdrawn axially from the plunger housing 135.

In some embodiments, atomizer device 100 may include a proximal outlet tube assembly 320 having asymmetrical ports that are configured to be attached respectively to the gas channel and liquid channel within the head unit 105. As shown, though without limitation, a first port has a first diameter and a second port has a second diameter, wherein the first diameter is less than the second diameter. It will be appreciated that the asymmetrical ports in either size, shape, orientation, etc. thus ensure proper assembly by the user and prevent misalignment of the gas channel and liquid channel with the atomizer nozzle 155. The second port may also serve as a fitting that retains spring 170. The proximal outlet tube assembly 320 may have a pair of angled tabs 322 projecting laterally from opposing sides thereof.

In some embodiments, atomizer device 100 may include a distal outlet tube assembly 325 comprising gas outlet tube 195 and liquid outlet tube 200. Distal outlet tube assembly 325 may feature a locking mechanism 330 configured to be secured to the angled tabs 322 along the sides of the proximal outlet tube 320. The locking mechanism 330 may feature flexible pinch tabs and hooks that may be extended over and then secured to the angled tabs 322 on the proximal outlet tube to secure the two pieces to one another. It will be appreciated that use of a proximal outlet tube assembly 320 that is releasably couplable to distal outlet tube assembly 325, as opposed to use of a single monolithic structure, enables the user to detach and discard the distal outlet tube assembly 325 and reuse the rest of the atomizer device 100. In practice, a user may detach the locking mechanism 330 from the angled tabs 322 to release the distal outlet tube assembly 325 from the proximal outlet tube assembly 320 and thereafter discard and replace it with minimal to no sterilization required. This configuration further reduces the risk of cross-contamination between patients, ensuring a higher standard of hygiene and infection control.

Alternatively, as shown in FIGS. 11-12, in some embodiments, atomizer device 100 may feature an outlet tube assembly 125 only, instead of having both a distal and proximal outlet tube assembly as discussed above. Such embodiments may be suited for single-use, disposable applications. Referring again to FIGS. 10-13, in some embodiments, the atomizer nozzle 155 includes a body 335 and cap 340, which are coupled together using any suitable manner. In some embodiments, body 335 and cap 340 are coupled together using an interference fit, a friction fit, a snap fit, threads, sonic weld, etc. In one or more embodiments, adhesive is applied to body 335 and/or cap 340 before being pressed together, ensuring a permanent or semi-permanent coupling. In some embodiments, a friction fit, interference fit, sonic weld, or snap fit is used without adhesive to allow different caps 340 and bodies 335 to be used together.

FIG. 14 is a sectional view of atomizer nozzle 155 in accordance with one or more embodiments of the present disclosure. As shown, the body 335 may include a terminal gas channel 345 adjacent to a terminal liquid channel 350, wherein the terminal gas channel 345 narrows towards an ending thereof while the terminal liquid channel 350 remains uniform in diameter throughout. Both the terminal gas channel 345 and terminal liquid channel 350 transition to a mixing region 355 or reservoir having a frustrum 360 (e.g., conical outlet) whereby liquids which may include medicament exit the atomizer nozzle 155. When pressurized gas flows through the terminal gas channel 345, the narrowing geometry at the ending thereof creates a Venturi effect, reducing static pressure in the mixing region 355. This pressure drop draws liquid from the terminal liquid channel 350 into the gas stream without requiring additional pumping. The high-velocity gas shears the liquid into fine droplets, forming an atomized mist. The geometry of the channels 345, 350, including their relative alignment, tapering, and intersection angle, ensures efficient entrainment of liquid and promotes droplet breakup. The resulting mist exhibits a uniform particle size distribution suitable for applications of medicament.

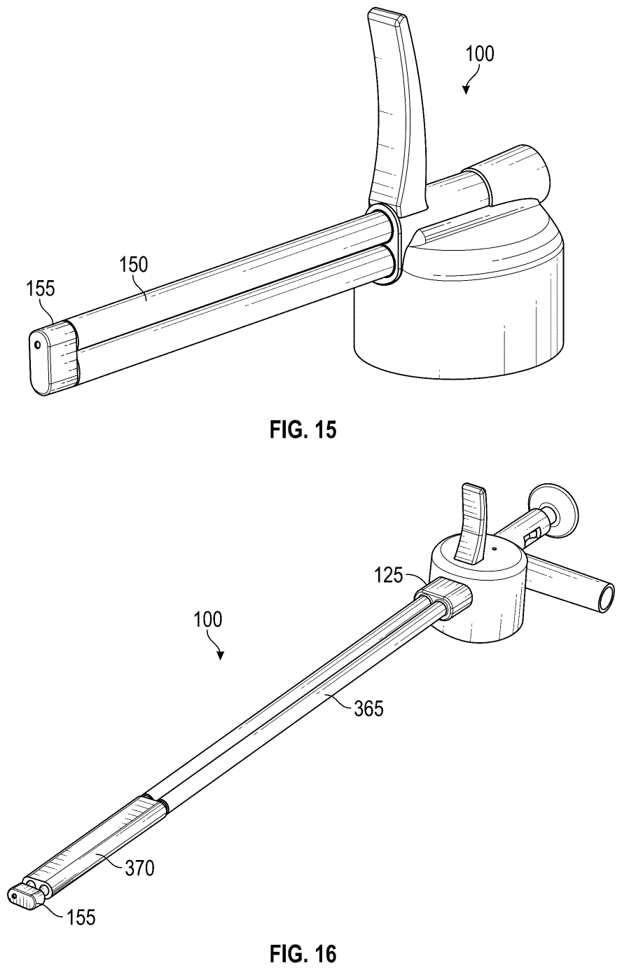

FIG. 15 is a perspective view of atomizer device 100, in accordance with one or more embodiments of the present disclosure. As shown therein, the nozzle extension 150, as discussed earlier may extend between the outlet tube assembly (not shown) towards the atomizer nozzle 155. As shown, the nozzle extension 150 may be rigid. The rigidity can be accomplished via use of medical-grade rigid plastics (e.g., polycarbonate, polypropylene, etc.), stainless steel for high durability and sterilization compatibility, or other rigid materials in the tubing. The wall thickness of the nozzle extension 150 is sufficient to resist bending under normal operating pressure. Whereas ENT procedures often require accurate delivery of medication or saline mist to specific anatomical areas such as the nasal cavity, sinuses, or throat, it will be appreciated that rigid tubing maintains its shape, allowing the clinician to aim the atomizer device 100 without the tubing bending or collapsing. Rigid tubing ensures a stable internal diameter, preserving airflow and atomization quality. A rigid tube acts like an extension of the atomizer device 100, making it easier to maneuver without needing adjustments. As depicted, the orientation of the nozzle extension 150 may be vertical as an alternative to a horizontal design.

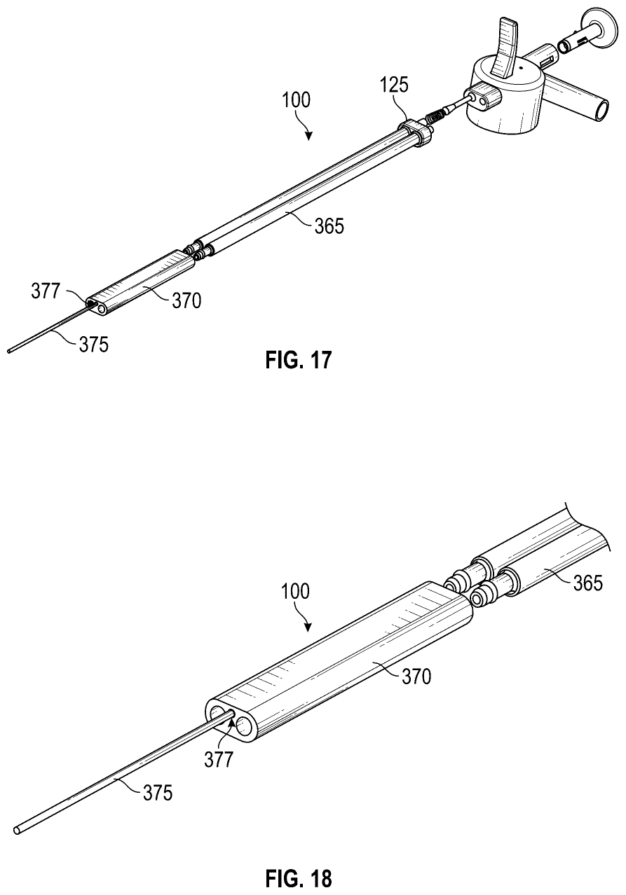

FIGS. 16-17 are respectively a perspective view and an exploded view of atomizer device 100, in accordance with one or more embodiments of the present disclosure. The atomizer device 100 in said embodiments may feature an outlet tube assembly 125 coupled to a fixed extension member 365. The fixed extension member 365 is rigid and immovable. Current atomizers available today require a clinician to position their hands either on or otherwise uncomfortably close to the patient's face when inserting the atomizer within the patient's mouth or nose. Having a fixed extension member 365 provides additional distance for the convenience of the clinician and comfort of the patient during such procedures. The fixed extension member 365 is selectively couplable to an adjustable extension member 370 at a distal end thereof which in turn is selectively couplable to an atomizer nozzle 155.

FIG. 18 is an enlarged view of the adjustable extension member 370. The adjustable extension member 370 may comprise an elongated body having an internal lumen 377 along its longitudinal axis. As best seen here, a wire 375 may be disposed within the lumen 377 to impart a structural rigidity that enables the user to electively adjust the adjustable extension member 370 and form bends, loops, or angled sections that facilitate navigation through anatomical pathways. The pairing of the fixed extension member 365 together with the adjustable extension member 370 permits the clinician to have further reach down difficult to access regions like the nasal cavities and throat while also providing an angled tip at the end with range of motion from which to direct the atomized fluids and provide targeted delivery of medicament. In some embodiments, the atomizer device 100 may be fully disposable and, in such embodiments, may therefore remove certain locking mechanisms between the pieces thereof for simplicity.

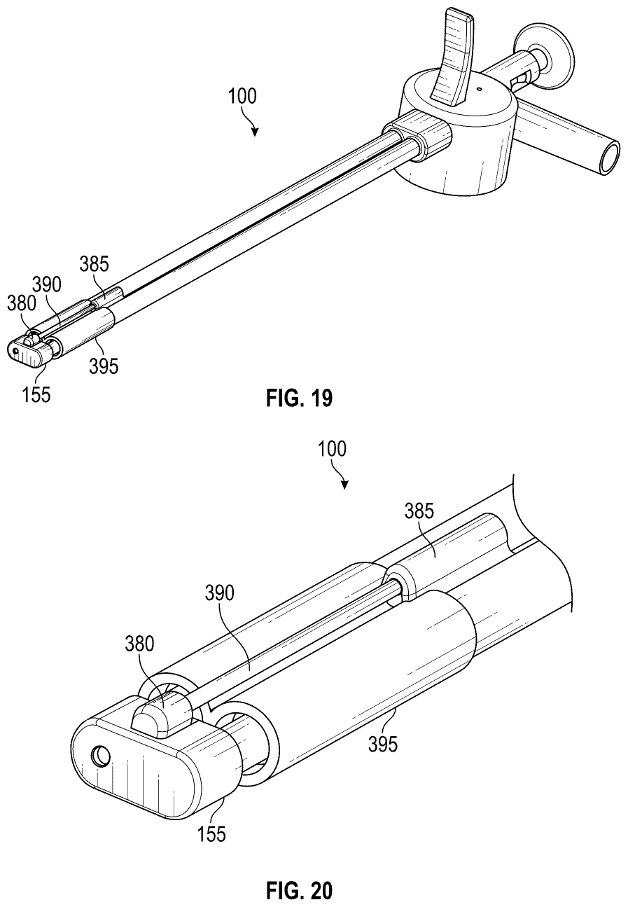

FIGS. 19-20 are respectively a perspective view of atomizer device 100 and an enlarged view of the tip thereof, in accordance with one or more embodiments of the present disclosure. In such embodiments, atomizer device 100 may include a distal mount 380 on the atomizer nozzle 155 and a proximal mount 385 on the fixed extension member 365 or other portion of the nozzle extension and tubing. The distal mount 380 is aligned with the proximal mount 385 along a longitudinal axis of the fixed extension member 365 or nozzle extension, such that a wire segment 390 may be inserted between the distal mount 380 and the proximal mount 385. An adjustable member 395 may extend from the fixed extension member 365 to the atomizer nozzle 155, substantially along the length of the wire segment 390. The wire segment 390 enables the clinician or other user of the atomizer device 100 to selectively shape and position the wire segment 390 and the adjustable member 395 substantially along the length thereof. The atomizer 100 may thus be configured to retain a bent configuration during use in ear, nose, and throat procedures.

Accordingly, the atomizer device disclosed herein addresses the problems in the prior art by providing a design with fewer components, enhanced safety, compatibility with various gas sources, and adjustable tips, all while achieving efficient atomization at lower supply pressures.

1The present disclosure may be embodied in other specific forms without departing from its structures, methods, or other essential characteristics as broadly described herein and claimed hereinafter. The described embodiments are to be considered in all respects only as illustrative, and not restrictive. The scope of the disclosure is, therefore, indicated by the appended claims, rather than by the foregoing description. All changes that come within the meaning and range of equivalency of the claims are to be embraced within their scope.

1. An atomizer device, comprising:
a head unit having a proximal end and a distal end, the proximal end configured to receive a plunger and to receive a gas flow through a gas inlet, wherein the plunger is configured to actuate a valve that opens to allow a gas to flow through a gas channel in the head unit;
a container receptacle coupled to the head unit configured to hold a liquid; and
an atomizer nozzle coupled to the distal end of the head unit and configured to mix the gas and the liquid together.

2. The device of claim 1, wherein the atomizer nozzle utilizes the venturi effect to draw the liquid from the container receptacle and mix the gas and the liquid together forming a fine spray mist.

3. The device of claim 1, wherein the valve comprises a tapered outer surface of a valve needle and a tapered inner surface of the gas channel, wherein the tapered inner surface is configured to press against the tapered outer surface when closed to seal the gas channel.

4. The device of claim 3, wherein the head unit comprises a liquid channel in fluid communication with the container receptacle and the atomizer nozzle.

5. The device of claim 1, further comprising a spring configured to bias the valve to a closed position.

6. The device of claim 1, wherein the atomizer nozzle comprises a nozzle extension, wherein the nozzle extension comprises a gas lumen, a liquid lumen, and a third lumen with a wire disposed therein.

7. The device of claim 1, wherein the head unit comprises a finger support on a top of the head unit.

8. The device of claim 1, wherein the plunger further comprises tabs configured to be couplable to windows on a plunger housing.

9. The device of claim 1, further comprising a wire segment couplable between a distal mount on the atomizer nozzle and a proximal mount on the nozzle extension, configured to retain a bent configuration.

10. An atomizer device consisting essentially of:
a head unit comprising a cap having an internal threaded cavity, a sidewall of the cap having a proximal end, a distal end, and a gas channel extending therebetween, the proximal end comprising a plunger housing in fluid communication with the gas channel, the plunger housing further comprising a gas inlet in fluid communication with the gas channel, and the distal end further comprising a liquid channel in fluid communication with the internal threaded cavity;
a valve needle slidably disposed within the gas channel such that a proximal end of the valve needle is positioned within plunger housing;
a spring positioned within the gas channel between a distal end of the valve needle and the distal end of the head unit;
a plunger slidably disposed within the plunger housing, a body of the plunger having a slot extending along a length of the body;
a securement pin inserted through the plunger housing and the slot of the plunger such that the securement pin retains the plunger within the housing; and
an outlet tube assembly having a gas outlet tube coupled to the gas channel at the distal end of the head unit, and a liquid outlet tube coupled to the liquid channel at the distal end of the head unit.

11. The device of claim 10, wherein the outlet tube assembly is configured to receive a nozzle extension.

12. The device of claim 10, wherein a top surface of the head unit further comprises a finger support.

13. The device of claim 10, wherein the valve needle comprises a tapered outer surface configured to contact a tapered inner surface of the gas channel to selectively prevent a gas from flowing through the gas channel.

14. A method of assembling the atomizer device of claim 10, comprising:
disposing the valve needle into the gas channel of the head unit;
inserting the spring into the gas channel such that the spring is positioned between the disposed valve needle and the distal end of the head unit;
securing the outlet tube assembly to the gas channel and the liquid channel at the distal end of the head unit;
disposing the plunger in the plunger housing and temporarily holding the plunger in a depressed position within the plunger housing and securing the securement pin through the plunger housing and the slot of the depressed plunger.

15. The method of claim 14, further comprising applying either an adhesive or sonic welding joint between the outlet tube assembly and the head unit.

16. The method of claim 15, further comprising releasing the depressed plunger after the securement pin is secured to the plunger housing.

17. The method of claim 14, further comprising coupling a nozzle extension to the outlet tube assembly.

18. The method of claim 17, wherein the nozzle extension comprises a lumen for securing a wire such that the nozzle extension is configured to retain a bent configuration.

19. The method of claim 14, wherein the outlet tube assembly further comprises a nozzle extension and an atomizer nozzle.

20. The method of claim 14, wherein the nozzle extension and or atomizer nozzle are detachable and replaceable.
